(19) United States
(12) Patent Application Publication
(10) Pub. No.: US 2022/0117792 A1
(43) Pub. Date: Apr. 21, 2022

(54) GAUZE FOR TOPICAL APPLICATION ON A TARGET AREA AND A PACKAGING THEREOF

(71) Applicant: 4B Ventures LLC, Dripping Springs, TX (US)

(72) Inventor: Caroline Bradford, Dripping Springs, TX (US)

(73) Assignee: 4B Ventures LLC, Dripping Springs, TX (US)

(21) Appl. No.: 17/076,147

(22) Filed: Oct. 21, 2020

(51) Int. Cl. A61F 13/00

(52) U.S. Cl. CPC A61F 13/00029; A61F 13/00072; A61F 13/00017; A61F 13/00012; A61F 13/00063

(57) ABSTRACT

Disclosed is a gauze for topical application on a target area. The gauze comprises a fabric layer having a first side and a second side opposite to the first side, wherein the first side is arranged to face the target area; a lubricant layer applied on the first side of the fabric layer; and a peelable protective layer arranged on the lubricant layer and another peelable protective layer arranged on the second side of the fabric layer. Disclosed also is a gauze for post-operative care of a circumcision wound. Disclosed further is a package comprising a plurality of gauzes, wherein each of the plurality of gauzes is discretely wrapped in a wrapper.

### TECHNICAL FIELD

The present disclosure relates generally to medical dressings; and more specifically, to gauzes for topical application on a target area. Moreover, the present application relates to a post-operative care of a circumcision wound using the aforementioned gauze. Furthermore, the present application relates to a package comprising a plurality of gauzes.

### BACKGROUND

Medical advancements in surgeries and post-operative care of surgical wounds have opened avenues for developments in the area of surgical dressings. Typically, surgical dressings include gauzes, bandages, adhesive bandages, foams, hydrogel dressings, alcohol preps, and the like. However, specific surgical dressings are required for specific types of wounds for their post-operative care.

Male circumcision is one of the most common surgical procedures in the world. Male circumcision is essentially a personal, religious and cultural choice, where all or a part of the foreskin (or prepuce) that typically covers the head of the penis is surgically removed. Notably, the male circumcision is beneficial in that it prevents urinary tract infections, penile cancer, transmission of some sexually transmitted infections, including HIV. Despite the afore-mentioned merits, male circumcision is still associated with risks and acute complications in many cases, especially in case of newborns' circumcisions, and require extreme post-operative care of the circumcision wound.

Conventionally, the post-operative care of the circumcision wound requires application of a lubricant on the wound and covering it with a medical-grade wound dressing such as a gauze. Till date, it has been solved by using individual products, i.e. a gauze, a lubricant, a cleansing solution, an antibiotic ointment, and so on. However, such conventional way of post-operative care requires using a piece of gauze, not of any specifics, plus having to add the lubricant, such as a petroleum jelly, on one side of the gauze, all while making a bit of a mess. This is all the more stressful when performed for a newborn's circumcision wound, all while changing a newborn's diaper is not the easiest task.

Moreover, multiple gauzes are conventionally packed in a single wrapping sheet, such as a plastic film. Sterility of gauzes remaining in the pack is challenged when the pack is opened to use one of the packed gauzes. Often, such conventional gauzes impair the healing process and further infect the irregularly shaped and extremely deep wounds, essentially due to ineffective water- and infection barrier. Moreover, such dressings are compromised in absorbing exudate fluids from the wounds that further lead to necrosis or pus at the wound site as well as in its vicinity. Additionally, the conventional wound dressings adhere to the healing wound and damaging the wound during their removal from the wound.

Therefore, in light of the foregoing discussion, there exists a need to overcome the aforementioned drawbacks associated with the conventional wound dressings used in post-operative care of surgical wounds, such as circumcision wound, and provide an effective and user-friendly alternative to the conventional techniques.

### SUMMARY

The present disclosure seeks to provide a gauze for topical application on a target area. The present disclosure also seeks to provide a gauze for post-operative care of a circumcision wound. The present disclosure also seeks to provide a package comprising a plurality of gauzes. The present disclosure seeks to provide a solution to the existing problem of conventional medical dressings, that are both complex and prone to contamination from a non-sterile environment thereby contaminating a surgical wound. An aim of the present disclosure is to provide a solution that overcomes at least partially the problems encountered in prior art, and provides an independent, efficient, cost-effective and user-friendly all-in-one solution.

In addition to the problems as indicated above, the inventors have further realized that conventional medical dressings for post-operative care of circumcision wound, even if based on lubricant application to the wound, fail to efficiently heal or protect the wound from a potential contamination from a non-sterile external environment as well as tissue necrosis.

In one aspect, an embodiment of the present disclosure provides a gauze for topical application on a target area, the gauze comprising:
a fabric layer having a first side and a second side opposite to the first side, wherein the first side is arranged to face the target area;
a lubricant layer applied on the first side of the fabric layer; and
a peelable protective layer arranged on the lubricant layer and another peelable protective layer arranged on the second side of the fabric layer.

Optionally, the fabric layer is a non-woven fabric layer.

Optionally, the fabric layer is a woven fabric layer, and wherein the woven fabric layer comprises a metal-fabric composite layer.

Optionally, the fabric layer is composed of a natural fiber, a synthetic fiber, a metal-fabric composite fiber, or a combination thereof.

Optionally, the gauze is made from a multi-ply fabric layer.

Optionally, the gauze is made from a rayon and polyester blend comprised of 8-ply.

Optionally, the amount of lubricant layer is in a range between 3 and 20 grams.

Optionally, the lubricant layer is at least one of: petroleum jelly or medical-grade oil.

Optionally, the target area is a skin having any of: a post-operative wound, an injury or a skin condition.

In another aspect, an embodiment of the present disclosure provides a gauze for post-operative care of a circumcision wound, the gauze comprising:
a fabric layer having a first side and a second side opposite to the first side, wherein the first side is arranged to face the circumcision wound;
a lubricant layer applied on the first side of the fabric layer; and
a peelable protective layer arranged on the lubricant layer and another peelable protective layer arranged on the second side of the fabric layer.

Optionally, the fabric layer is a non-woven fabric layer.

Optionally, the gauze is made from a multi-ply fabric layer.

Optionally, the gauze is made from a rayon and polyester blend comprised of 8-ply.

Optionally, the amount of lubricant layer is in a range between 3 and 20 grams.

Optionally, the lubricant layer is at least one of: petroleum jelly or medical-grade oil.

In yet another aspect, an embodiment of the present disclosure provides a package comprising a plurality of gauzes, wherein each of the plurality of gauzes is discretely wrapped in a wrapper.

Optionally, the plurality of wrappers are fabricated from a material selected from: aluminium, plastic, paper, or any combination thereof.

Optionally, the package further comprises a bioactive component.

Embodiments of the present disclosure substantially eliminate or at least partially address the aforementioned problems in the prior art, and enables a less stressful post-operative care of a surgical wound, such as a circumcision wound.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following drawings wherein:

FIGS. 1A and 1B are illustrations of a gauze for topical application on a target area, according to one embodiment of the teachings herein;

FIGS. 2A and 2B are schematic illustrations of a packaging of a gauze discretely wrapped in a wrapper, in a closed and open configuration, respectively, according to one embodiment of the teachings herein;

FIGS. 3 to 10 are schematic illustration of an exemplary embodiment of a discretely packaged gauze, according to one embodiment of the teachings herein; and

FIG. 11 is a schematic illustration of a package comprising a plurality of gauzes, wherein each of the plurality of gauzes is discretely wrapped in a wrapper, according to teachings herein.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION

The following detailed description illustrates embodiments of the present application and ways in which they can be implemented. Although some modes of carrying out the present teachings have been disclosed, those skilled in the art would recognize that other embodiments for carrying out or practicing the present teachings are also possible.

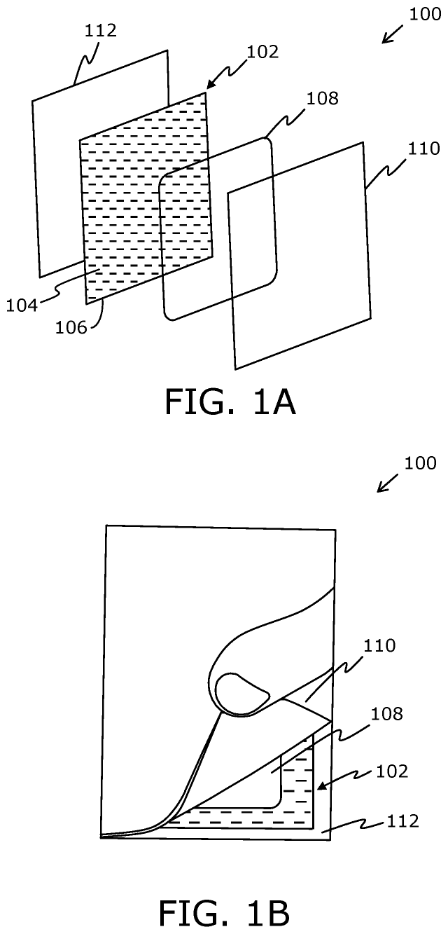

Referring to FIGS. 1A and 1B, shown are schematic illustrations of a gauze 100 for topical application on a target area (not shown). The gauze 100 comprises a fabric layer 102 having a first side 104 and a second side 106 opposite to the first side 104. The first side 104 is arranged to face the target area. Moreover, the gauze 100 comprises a lubricant layer 108 applied on the first side 104 of the fabric layer 102. Furthermore, the gauze 100 comprises a peelable protective layer 110 arranged on the lubricant layer 108 and another peelable protective layer 112 arranged on the second side 106 of the fabric layer 102.

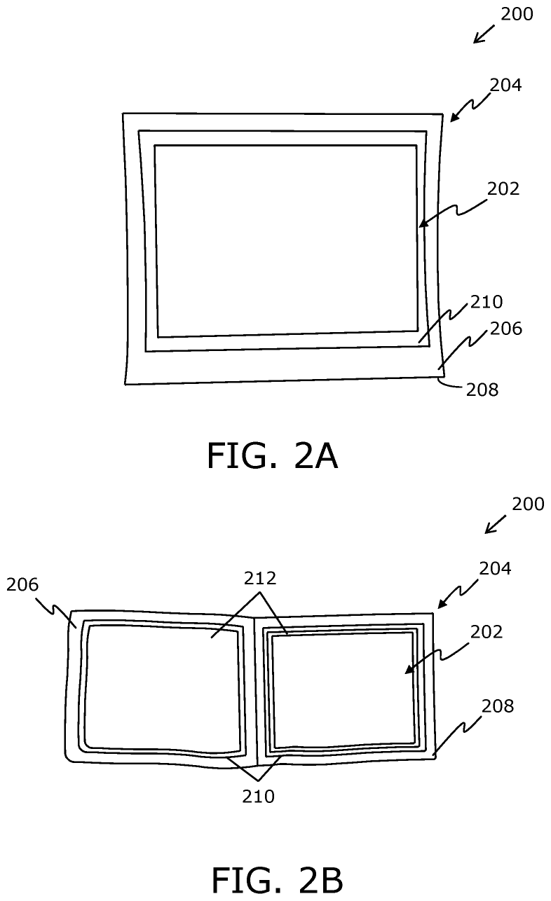

Referring to FIGS. 2A and 2B, shown is a packaging 200 of a gauze 202 discretely wrapped in a wrapper 204, in a closed and open configuration, respectively.

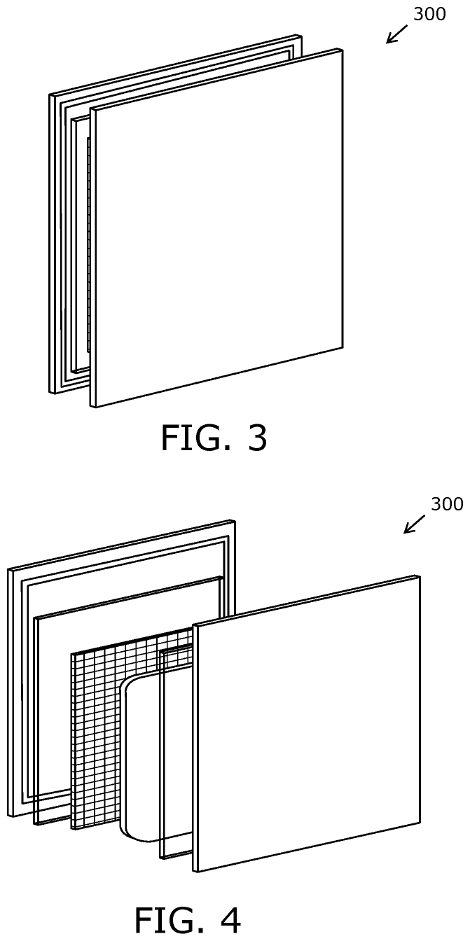

Referring to FIGS. 3 to 10, shown are schematic illustration of an exemplary embodiment of a discretely packaged gauze 300.

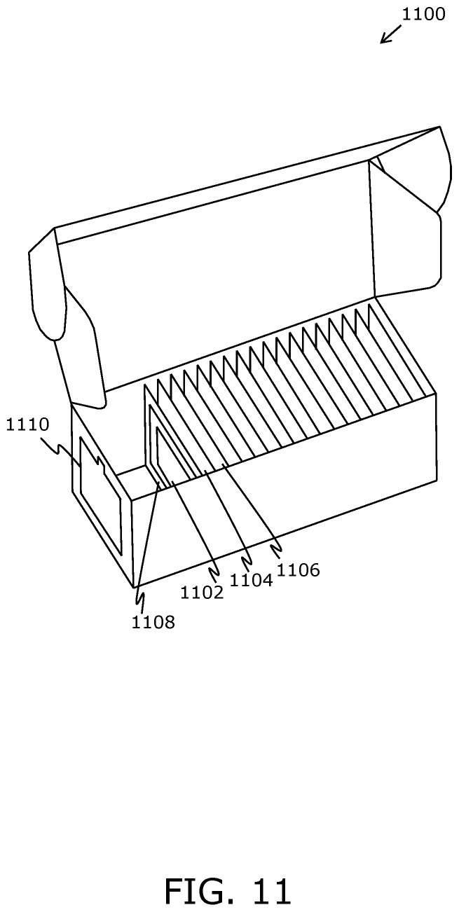

Referring to FIG. 11, shown is a schematic illustration of a package 1100 comprising a plurality of gauzes, such as gauzes 1102, 1104, 1106, wherein each of the plurality of gauzes, such as gauzes 1102, 1104, 1106, is discretely wrapped in a wrapper 1108.

The present disclosure provides the gauze for topical application on the target area, such as a surgical wound, for example, a circumcision wound. The gauze of the present disclosure is an independent single-product solution for post-operative care of a circumcision wound. The gauze is latex-free and composed of medical-grade and approved fibers for a no-irritation experience. Moreover, the fibers of the gauze are non-woven and joined together by mechanical setting, thereby making the gauze thin, soft, flexible, more durable, and with enhanced wicking and absorbent capacity. Beneficially, the flexibility of the gauze renders it readily available for application to wounds of irregular shape and depths. Furthermore, each gauze is a discretely (or individually) wrapped unit to allow application thereof during travelling and on-the-go purposes. Moreover, the lubricant layer atop of the gauze is a medical-grade lubricant that provides a contamination- and water-proof natural healing environment for the wound. Furthermore, the pre-lubricated gauze is a zero adhesive dressing and therefore can be conveniently removed as a single piece without sticking to the healing or healed wound. Additionally, the pre-lubricated non-woven gauze of the present disclosure is suitable to be applied to any type of wound, skin condition or any other surface without leaving lint or fibers at the target site after removal therefrom. Furthermore, the peelable protective layer protects the lubricant layer from drying out or attracting any contaminating particles. Additionally, the peelable protective layer offers a zero-mess application of the pre-lubricated gauze to the wound. Such an ‘all-in-one’ product offers a less stressful application for the post-operative care of a circumcision wound, especially in case of a newborn's circumcision. Beneficially, the gauze of the present disclosure provides an easy diaper and dressing change experience to the applicators, such as parents or medical providers, in case of a newborn's circumcision that takes up to 7 to 10 days' time to heal completely.

Examples of embodiments according to the teachings herein will now be described in detail with simultaneous reference to FIGS. 1, 2, and 3.

Throughout the present disclosure, the term “gauze” 100 as used herein refers to a light, open mesh of fibers for use as dressings and swabs. Specifically, the gauze 100 is a transparent or translucent fabric made of fibers running over each other in warp (longitudinally) and weft (perpendicular to warp) directions. Typically, gauze 100 is available in various sizes, shapes, and thicknesses. The gauze 100 offers a variety of options for example singly layer or multi-layer (referred to as “ply” hereafter), woven or nonwoven, sterile or non-sterile, plain or impregnated (coated or saturated), and perforated or slit. Beneficially, the arrangement of fibers in the gauze allows gases and water vapours to pass therethrough, while still serving as strong barrier against liquid water, microbes and dust particles.

The term “topical application” as used herein refers to applying directly to a surface or a particular part of a surface, such as for example a part of the body, i.e. skin. Specifically, topical application of the gauze 100 provides at least one of: covering, cleaning or treating of the target area external to a body (i.e., exposed). The term “target area” as used herein refers to the site or surface or the particular part of the surface, such as for example a part of the body, i.e. skin, for topical application of the gauze 100. The target area may typically be a site requiring medical attention. Optionally, the target area is a skin having any of: post-operative or surgical wound (for example, a circumcision wound, a necrotic wound, a clean wound, an infected wound, and so on), an injury or a skin condition (such as burn, rash, cut, bruise, scar, dryness, crack, soreness, itchiness, roughness, razor bump, and the like). Optionally, the target area is tissue beneath the skin. In this regard, the gauze 100 for topical application on the target area enables protecting the target area from injury and a potential infection, controlling bleeding and providing a moist environment for effective healing.

The term “fabric layer” 102 as used herein refers to a layer of flexible material consisting of natural or synthetic fibers (thread or yarn). Typically, the fibers are arranged to run across in perpendicular direction to form a weave pattern. Moreover, the fabric layer 102 of the present disclosure is typically a non-woven or very loosely woven fabric with open ends. The fibers are selected from a group comprising: natural fibers (such as cotton, linen, silk, hemp, wool, jute, flax, bamboo fibers, sea-weed fibers, mineral fibers and so on), synthetic fibers (such as rayon, nylon, polyester, polyurethane, polypropylene, acrylic, and so on), a metal-fabric composite fiber (such as silver fiber, gold fiber, and so on), or any combination thereof.

Optionally, the gauze 100 is a thin, soft and pliable single layer dressing made of the fabric layer 102. Optionally, the gauze 100 is a relatively thick pad-like structure made from multiple layers of thin, soft and multi-ply fabric layers 102. Optionally, the gauze 100 is made from a multi-ply fabric layer 102. The multi-ply fabric layer 102 is made of multiple fabric layers 102 layered on top of each other to provide increased absorbency, cushioning and thermal insulation. Optionally, the multi-ply fabric layer 102 ranges from 2 to 24 ply. The multi-ply fabric layer 102 is for example, a 2-ply, a 4-ply, a 6-ply, an 8-ply, a 10-ply, a 12-ply, a 16-ply, an 18-ply, a 20-ply, a 22-ply or a 24-ply fabric layer 102. In an example, the gauze 100 is composed of a fabric layer 102 having a length and a breadth of 12 inch and 12 inch respectively. The said fabric layer 102 is folded into a square-shape of a length and a breadth of 4 inch and 4 inch respectively to form a 4-ply gauze 100. In another example, the gauze 100 is composed of a fabric layer 102 having a length in a range of 2 inch to 12 inch and a breadth of 12 inch. The said fabric layer 102 is folded into a square-shape of a length and a breadth of 4 inch and 4 inch respectively to form a 8-ply gauze 100. Notably, the multi-ply arrangement of fabric layer 102 results in a first side 104 and a second side 106 opposite to the first side 104 of the fabric layer 102. The first side 104 of the fabric layer 102 is arranged to face the target area when in use.

Optionally, the fabric layer 102 is a non-woven fabric layer 102. Typically, the non-woven fabric layer 102 consists of fibers pressed together to resemble a weave. Optionally, the pressed together of fibers may be using at least one of: mechanical, chemical, thermal or solvent means. Optionally, the non-woven fabric layer 102 comprises any of: a spun-bond nonwoven fabric, a melt-blown nonwoven fabric or any combination thereof. Optionally, the non-woven fabric layer 102 is made of synthetic fibers, for example rayon, polyester, and so on. Optionally, the non-woven fabric layer 102 is made of a blend of natural and synthetic fibers, for example cotton and rayon, with a similar or varying concentration of the two types of fibers. Optionally, the non-woven fabric layer 102 is made of a blend of two or more synthetic fibers, for example rayon and polyester, with a similar or varying concentration of the different types of fibers. Beneficially, the non-woven fabric layer 102 provides improved wicking and absorbance thereby.

Alternatively, optionally, the fabric layer 102 is a woven fabric layer 102. Specifically, the woven fabric layer 102 has a loose, open weave (for example a plain weave) with greater absorbent capacity. Optionally, the woven fabric layer 102 is made of a fine or coarse mesh of cotton fibers or similar fibers selected from a group of natural fibers, synthetic fibers, or any combination thereof. Optionally, the woven fabric layer 102 comprises a metal-fabric composite layer, wherein the one type of fibers (for example, cotton fibers or similar fibers selected from a group of natural fibers, synthetic fibers, or any combination thereof) extending in the longitudinal direction, and the other type of fibers (for example, metal fibers) extending in the transverse direction.

Optionally, the gauze 100 is made from a rayon and polyester blend comprised of 8-ply. More optionally, the rayon and polyester blend comprises rayon fibers in a range of 20 to 90% and polyester fibers in a range of 20 to 90%. In an example, the rayon fibers are typically from 20%, 30%, 40%, 50%, 60%, 70% or 80% up to 30%, 40%, 50%, 60%, 70%, 80% or 90%, and the polyester fibers are from 20%, 30%, 40%, 50%, 60%, 70% or 80% up to 30%, 40%, 50%, 60%, 70%, 80% or 90%. Optionally, the rayon and polyester blend comprises rayon fibers and polyester fibers in a ratio of 50%:50%. Beneficially, the gauze 100 made of rayon and polyester blends are strong and soft. Additionally, beneficially, the non-woven fabric layer 102 produces less or no lint to protect the target area to have fewer fibers behind upon removal thereof.

Optionally, length of the gauze 100 is in a range between 2 and 8 inches (in) and the width of the gauze 100 is in a range between 2 and 8 in. The length of the gauze 100 may typically be from 2, 3, 4, 5, 6 or 7 in up to 3, 4, 5, 6, 7 or 8 in, and the width of the gauze 100 is typically from 2, 3, 4, 5, 6 or 7 inches up to 3, 4, 5, 6, 7 or 8 inches. Optionally, the gauze 100 is shaped as a square or a rectangle. In an example, the gauze 100 is shaped as a square, for example with dimensions of 2 in×2 in, 3 in×3 cm, 4 in×4 cm, 5 in×5 cm, 6 in×6 cm, 7 in×7 in or 8 in×8 cm. In another example, the gauze 100 is shaped as a rectangle, for example with dimensions of 2 in×4 cm, 3 in×5 cm, 4 in×6 cm, and so on.

Moreover, the gauze 100 comprises a lubricant layer 108 applied on the first side 104 of the fabric layer 102. The term “lubricant layer” as used herein refers to a substance introduced to reduce friction between surfaces in mutual contact by creating a slick surface. The lubricant layer 108 enhances moisture insulation (or moisture locking) at the target area. Notably, the lubricant layer 108 is applied on only one side of the gauze 100, preferably the first side 104, to faces the target area when in use, while leaving the opposite side, i.e. the second side 106, thereof dry for easy (zero mess) handling. It will be appreciated that the lubricant layer 108 is applied at the center of the gauze 100 leaving the edges thereof open for easy handling. Optionally, the lubricant layer 108 is at least one of: a petroleum jelly or medical-grade oil. More optionally, the petroleum jelly is a 100% petroleum jelly. Typically, the medical-grade oil is castor oil, one or more essential oils, and so on. Optionally, the lubricant layer 108 comprises a water-insoluble lubricant.

Optionally, the amount of lubricant layer 108 is in a range between 3 and 20 grams. The amount of lubricant layer 108 is typically from 3, 4, 5, 6, 7, 8, 9, 10, 11, 12, 13, 14, 15, 16, 17, 18 or 19 grams up to 4, 5, 6, 7, 8, 9, 10, 11, 12, 13, 14, 15, 16, 17, 18, 19 or 20 grams. It will be appreciated that the amount of the lubricant layer 108 may vary with the size of the gauze 100. In an example, a gauze 100 of dimensions 4 in×4 has approximately 14 grams of 100% white petroleum jelly. In another example, a gauze 100 of dimensions 4 in×4 has approximately 8 grams of 100% white petroleum jelly.

Optionally, the gauze 100 comprises suitable medical-grade products such as, for example, ointment, medicine, antibiotics, anesthetics, analgesics, germicides, antimicrobial agents, and the like. Optionally, the suitable medical-grade product may be applied on the gauze 100 based on a condition of the target area and prescription of a doctor. In this regard, the suitable medical-grade product provides its constitutional benefits and the lubricant layer 108 provides a barrier to water and infection and a fast-healing environment for the wound. Optionally, the gauze 100 is an impregnated gauze, coated or saturated with suitable medical-grade products.

Furthermore, the gauze 102 comprises a peelable protective layer 110 arranged on the lubricant layer 108 and another peelable protective layer 112 arranged on the second side 106 of the fabric layer 102. The term “pee/able” as used herein refers to an ability to separate to physically coupled surfaces in the course of opening (or exposing or presenting) a protected product without compromising integrity of either of the two surfaces. The peelable protective layer 110 and the peelable protective layer 112 are thin, non-opaque (i.e. transparent or translucent), non-permeable covering that is easy to remove, such as by peeling off, from a surface, such as lubricant layer 108 on the first side 104 of the fabric layer 102 or the second side 106 of the fabric layer 102, respectively. Additionally, the peelable protective layer 110 and the peelable protective layer 112 are designed strategically for making the arrangement (i.e. the gauze) convenient for use, easy to handle, and provide a controlled and aseptic means for application of the gauze 100. It will be appreciated that the peelable protective layer 110 is to be removed or peeled off, to expose the lubricant layer 108 to the target area when the gauze 100 is ready to be applied. Optionally, the peelable protective layer 112 may be removed or peeled off when the gauze 100 is applied at the target area. In an embodiment, the peelable protective layer 110 is capable of adhering to the lubricant layer 108 and typically protects the lubricant layer 108 from drying out or contaminating. Optionally, the peelable protective layer 110 and the peelable protective layer 112 are composed of a polymer material, cellulose, or any combination thereof. More optionally, the peelable protective layer 110 and the peelable protective layer 112 are composed of a material that is anti-microbial, thermally-stable and/or water-proof. Optionally, the peelable protective layer 110 and the peelable protective layer 112 are composed of polyethylene, polyvinyl chloride, high-impact polystyrene, polyvinyl alcohol, copolymers, and so on.

Moreover, in an aspect of the present disclosure, the gauze 100 is employed for post-operative care of a circumcision wound, similar to the gauze 100 for topical application on a target area. The term “circumcision wound” as used herein refers to a wound after surgical removal of all or part of the foreskin (or prepuce) that typically covers the head of the penis. It will be appreciated that circumcision is a personal, religious and cultural choice. The circumcision may be performed on a new-born baby boy or an adult male by techniques known in the art. The post-operative care of the circumcision wound includes inspection of site for bleeding, infection and ensuring wound healing. In this regard, an applicator is required to apply a gauze dressing to the circumcision wound. Optionally, the applicator is any of: a medical provider, a circumcised individual, or a family member thereof in case the circumcised individual is incapacitated, for example a new-born baby, a toddler or kindergartner.

The gauze 100 for post-operative care of a circumcision wound comprises a fabric layer 102 having a first side 104 and a second side 106 opposite to the first side 104, wherein the first side 104 is arranged to face the circumcision wound. The gauze 100 for post-operative care of a circumcision wound comprises a lubricant layer 108 applied on the first side 104 of the fabric layer 102. The gauze 100 for post-operative care of a circumcision wound comprises a peelable protective layer 110 arranged on the lubricant layer 108 and the peelable protective layer 112 arranged on the second side of the fabric layer. Optionally, the gauze 100 for post-operative care of a circumcision wound may be secured in place with a medical-grade tape or a cotton gauze wrap in case of a newborn circumcision if the newborn shows very active movements.

Optionally, the gauze 100 is sterile. Optionally, the gauze 100 is sterilized by application of any of: heat, one or more chemicals (selected from the group consisting of ethylene oxide gas, ozone, chlorine bleach, glutaraldehyde, formaldehyde, ortho-phthalaldehyde, hydrogen peroxide and peracetic acid), high pressure, filtration, autoclaving, and/or radiation (such as X-rays, gamma rays, UV light and/or subatomic particles). In an example, the sterilization is performed using ethylene oxide. Optionally, the gauze 100 is sterilized before or after its discrete packing (as shown in FIGS. 2A and 2B).

Optionally, a packaging 200 of a gauze 202 comprises discretely wrapping the gauze 202 in a wrapper 204. Optionally, the wrapper 204 is fabricated from a material selected from: aluminium, plastic, paper, or any combination thereof. The wrapper 204 comprises two wrapping sheets 206 and 208. The two wrapping sheets 206 and 208 are foil-lined paper sheets, wherein the foil 212 side of the two wrapping sheets 206 and 208 are arranged to face the gauze 202 when the latter is discretely packaged therein. The two wrapping sheets 206 and 208 are provided with an adhesive line 210 (shaped corresponding to the shape of the gauze 202) surrounding the foil 212 on each of the two wrapping sheets 206 and 208. The adhesive line 210 of the two wrapping sheets 206 and 208 is matched up over the gauze 202 thereby sealing the gauze 202 securely in the wrapper 204. Optionally, the paper side, opposite to the foil 212 side thereof, of the wrapper 204 is provided with instructions for the user. In an example, the instruction may be for example “Peel Here” directions in a corner to direct the user where to separate layers of the wrapper 204 to access the gauze 202. In another example, the instruction may correspond to the site for folding the gauze 202, when used on an uneven surface, such as for example a circumcision wound. Optionally, the wrapper 204 is sterile. Optionally, the wrapper 204 is sterilized before wrapping the gauze 202 or after wrapping. Optionally, the packaging 200 enables providing a sterile gauze 202 for each use. Optionally, a plurality of the packaging 200 is packed together in a sterile package (as shown in FIG. 3). Beneficially, the wrapper 204 provides easy-to-discard wrapping without leaving a lot of trash behind, especially while travelling.

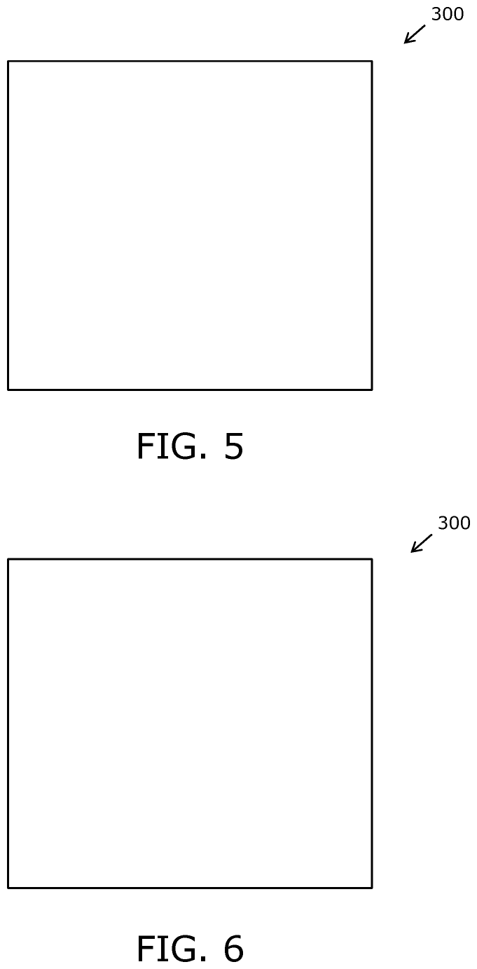

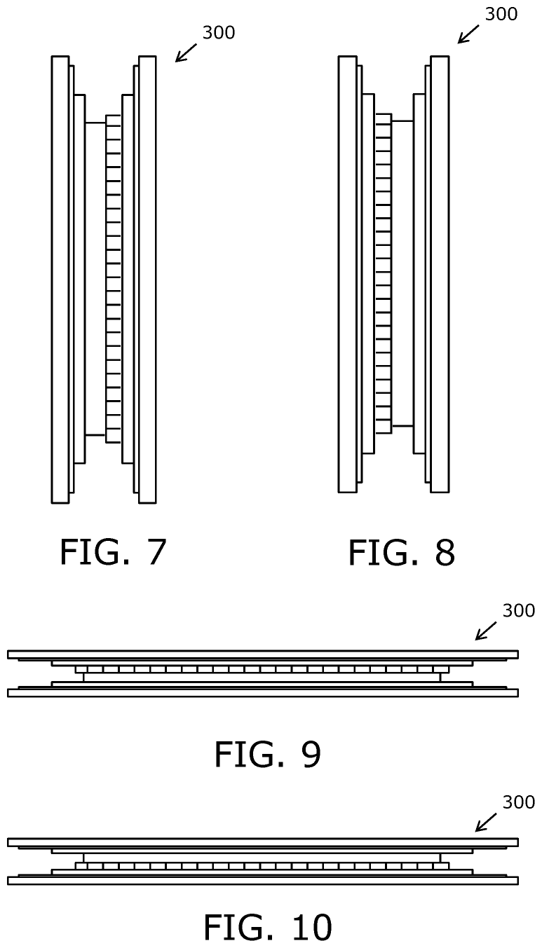

In an embodiment, the discretely packaged gauze 300 comprises a gauze similar to the gauze 100 of FIG. 1 or gauze 202 of FIG. 2. FIG. 3 is a perspective view of a discretely packaged gauze 300. FIG. 4 illustrates an exploded view of the of FIG. 3. FIGS. 5 and 6 illustrate a front view and a rear view of the of FIG. 3, respectively. FIGS. 7 and 8 illustrate a right side view and a left side view of the of FIG. 3, respectively. FIGS. 9 and 10 illustrate a top view and a bottom view of the of FIG. 3, respectively.

Moreover, the package 1100 comprises a plurality of gauzes, such as 1102, 1104, 1106, wherein each of the plurality of gauzes, such as 1102, 1104, 1106, is discretely wrapped in a wrapper 1108. Optionally, the package 1100 is implemented as a packing unit comprising a plurality of gauzes, such as 1102, 1104, 1106, with each of the plurality of gauzes, such as 1102, 1104, 1106, is discretely wrapped in a wrapper 1108.

Optionally, the package 1100 typically contains 10-30 discretely wrapped gauzes, such as 1102, 1104, 1106. In an example, the package 1100 contains 10 discretely wrapped gauzes, such as 1102, 1104, 1106. It will be appreciated that the package 1100 may be scaled to contain lesser or a greater number of discretely wrapped gauzes, such as 1102, 1104, 1106. It will further be appreciated that the size of the package 1100 may be scaled based on the number and size of the discretely wrapped gauzes, such as 1102, 1104, 1106.

Optionally, the length, width, and height of the package 1100 ranges from 2 to 15 in, 2 to 8 in, and 2 to 8 in, respectively. In an example, the length, width, and height of the package 1100 is typically from 2, 3, 4, 5, 6, 7, 8, 9, 10, 11, 12, 13 or 14 in up to 3, 4, 5, 6, 7, 8, 9, 10, 11, 12, 13, 14 or 15 in, 2, 3, 4, 5, 6, 7 or 8 in up to 3, 4, 5, 6, 7 or 8 in, and 2, 3, 4, 5, 6, 7 or 8 in up to 3, 4, 5, 6, 7 or 8 in, respectively. In an example, the length, width, and height of the package 1100 containing 30 units of a 4 in×4 in discretely wrapped gauzes, such as 1102, 1104, 1106, is 12 in, 4.2 in, and 4.2 in, respectively.

Optionally, the package 1100 is provided with an access area 1110 for accessing the discretely wrapped gauzes, such as 1102, 1104, 1106, individually. Notably, the access area 1110 enable accessing a single discretely wrapped gauze, such as 1102, 1104, 1106, without opening the entire package 1100 and exposing all the discretely wrapped gauzes, such as 1102, 1104, 1106, to a potentially non-sterile environment. Optionally, the access area 1110 is protected by a removably replaceable seal (not shown). Optionally, the package 1100 is provided with instruction for a user (such as for example the applicator). In an example, the instruction may read “PINCH HERE” to denote the site for pulling out single discretely wrapped gauze, such as 1102, 1104, 1106.

Optionally, the package 1100 further comprises a bioactive component. Optionally, the bioactive component is selected from the group consisting of: antihemorrhagics (such as coagulation factors (Factors V, Va, VII, VIIa, VIII, VIIIa, IX, IXa, X, Xa, XI, XIa, XII, XIII and/or XIIIa), fibrinogens, Vitamin K, antifibrinolytics, and so on), wound healing stimulants, infection antagonists, endothelium Tissue Factor (TF), tissue factor pathway stimulants, thrombin, plasmin, prothrombinase complex, Willebrand factor (vWF), tenase complex, fibrin strands formation stimulants, fibrin cross-linking stimulants, platelet aggregation stimulants, tranexamic acid, contact activation pathway stimulants, coagulation cascade stimulants, and so on. In an example, the bioactive component is an antibiotic ointment to add to the gauzes, such as 1102, 1104, 1106, if the surgical wound, such as a circumcision wound, shows any signs of early infection. Optionally, the bioactive component is supplied in a tube, a sachet, a vial, and the like. More optionally, the size of the bioactive component packaging conforms to the size of the package 1100.

Notably, the gauze 100 is used in medical application as any of: a patch, a swab, a bandage, a wound dressing, a tissue dressing. However, besides use thereof in medical applications as mentioned above, the gauze 100 may be used for a wide range of non-medical applications. The different non-medical applications of the gauze 100 includes, but is not limited to, skin care (such as skin moisturization, soothing sunburn or rashes, removal of make-up or paint, highlighter, preventing scars, protect newly healed tattoos or piercings, heal skin conditions for example dryness, soreness, itchiness, roughness, eczema, cracks, scrapes or bumps), and help for pet paws), coating on objects (such as metal hardware to prevent rusting, lubricating stuck objects, jewellery care, removing scuff on leather boots, shoes, bags, and handbags by buffing with lubricated gauze, and polish for leather clothes, shoes and furniture), and water-barrier (for wounds, ear-fittings for swimmers and flame producer for grills and fire-pits). Beneficially, the gauze 100 provides a stand-alone solution (i.e. an “all-in-one” product) eliminating the need for multiple products, such as a gauze, a pack of lubricant, and so on, for arriving at a same solution, i.e. a medical or non-medical application.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A gauze for topical application on a target area, the gauze comprising:
a fabric layer having a first side and a second side opposite to the first side, wherein the first side is arranged to face the target area;
a lubricant layer applied on the first side of the fabric layer; and
a peelable protective layer arranged on the lubricant layer and another peelable protective layer arranged on the second side of the fabric layer.

2. The gauze of claim 1, wherein the fabric layer is a non-woven fabric layer.

3. The gauze of claim 1, wherein the fabric layer is a woven fabric layer, and wherein the woven fabric layer comprises a metal-fabric composite layer.

4. The gauze of claim 1, wherein the fabric layer is composed of a natural fiber, a synthetic fiber, a metal-fabric composite fiber, or a combination thereof.

5. The gauze of claim 1, wherein the gauze is made from a multi-ply fabric layer.

6. The gauze of claim 1, wherein the gauze is made from a rayon and polyester blend comprised of 8-ply.

7. The gauze of claim 1, wherein the amount of lubricant layer is in a range between 3 and 20 grams.

8. The gauze of claim 1, wherein the lubricant layer is at least one of: petroleum jelly or medical-grade oil.

9. The gauze of claim 1, wherein the target area is a skin having any of: a post-operative wound, an injury or a skin condition.

10. The gauze for post-operative care of a circumcision wound, the gauze comprising:
a fabric layer having a first side and a second side opposite to the first side, wherein the first side is arranged to face the circumcision wound;
a lubricant layer applied on the first side of the fabric layer; and
a peelable protective layer arranged on the lubricant layer and another peelable protective layer arranged on the second side of the fabric layer.

11. The gauze of claim 10, wherein the fabric layer is a non-woven fabric layer.

12. The gauze of claim 1, wherein the gauze is made from a multi-ply fabric layer.

13. The gauze of claim 10, wherein the gauze is made from a rayon and polyester blend comprised of 8-ply.

14. The gauze of claim 10, wherein the amount of lubricant layer is in a range between 3 and 20 grams.

15. The gauze of claim 10, wherein the lubricant layer is at least one of: petroleum jelly or medical-grade oil.

16. The package comprising a plurality of gauzes, wherein each of the plurality of gauzes is discretely wrapped in a wrapper.

17. The package of claim 15, wherein the plurality of wrappers are fabricated from a material selected from: aluminium, plastic, paper, or any combination thereof.

18. The package of claim 15, wherein the package further comprises a bioactive component.
